(19) United States
(12) Patent Application Publication
(10) Pub. No.: US 2009/0149732 A1
(43) Pub. Date: Jun. 11, 2009

(54) System for use of electrical resonant frequencies in analyzing and treating abnormality of human and animal tissues

(76) Inventor: Ronald J. Weinstock, Corona Del Mar, CA (US)

Correspondence Address: MELVIN K. SILVERMAN AND ASSOCS PC, 500 WEST CYPRESS CREEK ROAD, SUITE 350, FT. LAUDERDALE, FL, 33309, US

(21) Appl. No.: 12/315,920

(22) Filed: Dec. 8, 2008

Related U.S. Application Data
(60) Provisional application No. 61/005,808, filed on Dec. 8, 2007.

(51) Int. Cl. A61B 5/055

(52) U.S. Cl. 600/407

(57) ABSTRACT

A system for use of electrical resonant frequencies in analyzing and treating abnormality of human and animal tissue includes elements for generating an EM wave having a selectable ratio of capacitance to inductance, modulated in a selectable electrical resonance (ER) frequency relative to the tissue, the tissue to be treated across an EM spectrum of between about 10 Hz and about 500 MHz; elements for measuring resultant ER phases and peaks of a responsive signal emitted by the tissue, in which each peak represents a potential in-phase or out-of-phase ER relative to an established normal phase of such tissue, each ER defining a waveform in which out-of-phase ER is associated with a tissue abnormality; and elements for delivery and applying to the tissue a waveform substantially inverse to that of out-of-phase waveforms to decrease or nullify ER peaks associated with the abnormality of tissue.

### CROSS REFERENCE TO RELATED APPLICATION

This application claims the benefit under 35 USC 119(e) of the provisional patent application Ser. No. 61/005,808, filed Dec. 8, 2007, which is hereby incorporated by reference in its entirety.

### BACKGROUND OF THE INVENTION

A. Area of Invention

The present invention relates to electromedicine and, more particularly, to the application of electrical resonant frequencies to tissue and the subsequent measurement of electromagnetic resonance properties of the tissue to ascertain and treat abnormalities associated therewith and with specific disease states.

B. Prior Art

The greater the time domain differences between the electrical and magnetic components of an electromagnetic (“EM”) response wave of a tissue, the greater will be the phase differences between those components and, thereby, the greater the energy loss. As such, measurement of differences in phase response has developed as a means of recognizing differences of properties between respective materials subject to EM waves of selectable frequencies. This phase change and energy loss relates loss of exponentially to cellular and tissue function.

From the perspective of quantum mechanics, the electron, while shifting positions within a permissible set of patterns relative to the atomic nucleus, generates specific energy emissions or spectra, i.e., electrical resonance (“ER”) by body tissues and physiological structures. Such patterns have been found to comprise unique tissue signatures and, as such, a product of the individual atomic and molecular quantum changes which characterize the given tissue, organ or physiological structure of interest. Thereby, from the perspective of biophysics, pathology emanating from a dysfunctional, damaged or diseased tissue is considered a misfunction of the LC equivalent circuit of the normal electron clouds associated with such tissue. ER disorders thereby predispose biochemical and bioelectrical alterations reflected in each unique ER signature of the tissue or structure of interest. When a disorder of measurable ER occurs, the resonance and configuration of the normal electron cloud at the atomic level exhibit phase shifts or frequency changes which break and disturb the otherwise orderly pathways of communication from atom to molecule, molecule to cell, cell to tissue, and tissue to organ. This results in breakdown in molecular and cellular communication, one end result being a reduction in tissue conductivity.

For the inventive electrical resonance analyzer and treatment system to function, the system sends or receives information to and from the body, the body's cellular network or, in some cases, the central nervous system, that is, its pain processing center. This pathway allows the ER of the inventive system to record and analyze any patterns that are ER phase or frequency-shifted, relative to established baselines, emanating by and from body tissues and structures.

In case of assessing and treating pain, the inventive system also employs inductors and applicators at the site of pain, tissue abnormality and/or upon selected nervous system trigger or motor points (which can exist at acupuncture or pressure points). A synthesized ER pattern is transmitted into the tissue which encounters the inherent RC circuit produced by the tissue or subject matter under study and a resonant signal is produced. The information generated by this initial step of the analysis process is returned to the system where, after removal of impedance or other static in the signal, it is analyzed, digitized and, if desired, compared to a database of predetermined ER patterns associated with normal tissue of the type of interest. Thereby, the received data is assessed and evaluated for irregulates or abnormalities. The inherent ER signatures of normal atoms, molecules, tissues and structures may thereby be employed as “standards” in the digitizing of values based on recorded and peak resonance emissions of healthy, non-diseased, non-damaged tissues. When such a first phase of the ER pattern measurement and assessment is complete, the system detects any disorder or shifting of ER peaks or changes from their normal frequency or frequency or phase shift, or frequency shape at the pain site under study, if such exists.

A second aspect of operation of the inventive system is that of its therapeutic action. If an ER phase abnormality of the targeted tissue or organ is detected in the first aspect of analysis, the shift can be corrected through the application of a counter or neutralizing ER which, as set forth below, is calculated and computed by the system, thereby resulting in a neurotransmitter function which is regulated by administration of a counter ER pattern. It has been found that upon realignment or normalization of the phase shifted ER pattern, reduction and alleviation of pain occur instantaneously, healing time is reduced and, upon suitable repetition of therapy can result in long term improvement of the abnormality of interest.

In terms of mechanism of operation of the invention, pain is reduced or eliminated by means of effect on nociceptive afferent neurons which are sensitive to EM as well as a variety of noxious stimuli including thermal, mechanical, and chemical means. Excitation of nociceptive neurons infer voltage as well as magnetic sensitive ion channels, particularly sodium, calcium and potassium channels. Nerve terminal membranes are EM encoded through the activation of inward depolarizing membrane currents or activation of outward currents. The main channels responsible for inward membrane currents are the voltage activated sodium and calcium channels.

It is known that an ionic gradient exists across the plasma membrane of virtually all human and animal cells. In particular, the concentration of potassium ions inside the cell is about ten times that in the extracellular fluid. Also, sodium ions are present in much higher concentrations outside a cell than inside. As such, the potassium and sodium channels play an important role in membrane excitation and, thereby, in determining the intensity of pain. Sodium channels are now considered a destabilizing membrane in the pain process. These channels, which can open rapidly and transiently when the membrane is depolarized beyond about minus 40 mV, are essential for action of most neurons, potential generation, and conduction. These open channels are also believed to be responsible for the neuron action leading to pain. Sodium is also an alkali element (with an atomic number of 11) and is paramagnetic. It is believed that an EM interaction thereby occurs between sodium channels and the ER patterns, phases and peaks discussed above. ER affects sodium which in turn affects the excitability of nociceptive neurons which are chemically distinct from most other neurons. It has been found that ER fields which consist of an RF carrier of a range of about 10 Hz to about 500 MHz provide a regulating effect upon sodium channels, this leading to pain reduction.

Tests have also indicated that ER fields alter the pH level of water, which relates to another theory of the pain reduction associated with the present system. That is, it has been shown that the pH of extra-cellular fluid is associated with a number of patho-physiological conditions such as hypoxia/anoxia and inflammation. It has been reported that the pH of synovial fluid from enflamed joints is significantly more acid than is that of normal joints. As such, low pH solutions evoke a prolonged activation of sensory nerves and produce a sharp stinging pain. Consequently, when pH of tissue is changed, pain reduction is often achieved.

Successful treatment of arthritis, fibromyalgia, neuralgia, neuropathy, categories of joint and tissue injury, wound healing, calcific tendonitis, and various types of migraine headaches has been demonstrated.

Prior art known to the inventor includes patents to Pilla, namely, U.S. Pat. No. 5,584,863 (1996), entitled Pulse Radio Frequency Electrotherapeutic System; and U.S. Pat. No. 5,723,001 (1998), entitled Apparatus and Method For Therapeutically Treating Human Body Tissue With Electromagnetic Radiation. Said patents teach improvements of TENS technology and o not address any form of LC electrical resonance in a therapeutic use. Pilla's earliest patent is U.S. Pat. No. 4,315,503, entitled Modification of the Growth, Repair, Maintenance and Behavior of Living Tissues and Cells by Specific and Selective Change in Electrical Environment

Schuler and Lee hold U.S. Pat. Nos. 6,633,779; 6,775,573; and 7,058,446 (enclosed herewith) directed to electrical methods for the treatment of asthma; GI tract conditions, and endocrine issues. Schuler and Lee use different waveforms, frequencies, and amplitudes as a function of the part of the human anatomy they seek to treat. However, they do not mention electrical resonance of a target tissue as a part of a therapeutic strategy.

In a diagnostic application, U.S. Pat. No. 6,704,662 (2004) to Gulati teaches the use of quantum resonance interferometry. The same may be of value in setting-up a database LC of cellular and tissue responses. U.S. Pat. No. 6,751,506 (2004) to Shealy employs electrical stimulation to reduce levels of free radicals.

U.S. 2005/0158285 (2005) to Giampapa teaches that electromagnetic signals may be used to modify the response of adult stem cells in order to simulate an embryonic behavior thereof.

U.S. Pat. No. 7,092,760 (2006) to Foster, entitled Electrical Stimulation of Tissue for Therapeutic and Diagnostic Purposes reflects an upgrade in TENS technology. Foster uses electrodes applied to the skull, apparently to monitor brain waves associated with pain and then to provide voltage bursts to neutralize “pain waves.” No reference is made to electrical resonance in the LC sense.

Another use by electric stimulation for healing acceleration, pain relief and destruction of pathogens (if any), appears in the enclosed U.S. Pat. No. 7,117,034 (2006) to Kronberg. This is a further upgrade of TENS technology.

U.S. Pat. No. 7,174,213 (2007) to Pless teaches the use of electrical stimulation, using various wave morphologies as a strategy for the prevention or reduction in the severity of waveforms of the brain associated with epilepsy.

It therefore is not known in the art to measure resonant EM frequencies and phase of tissue and to then treat the same with a generally inversely shaped and phased therapeutic wave based on the frequency and phase of the target tissue.

### SUMMARY OF THE INVENTION

A system for use of electrical resonant frequencies in analyzing and treating abnormality of human and animal tissue, the system comprising (a) means for generating an EM wave having a selectable ratio of capacitance to inductance, modulated in a selectable electrical resonance (ER) frequency relative to said tissue, the tissue to be treated across an EM spectrum of between about 10 Hz and about 500 MHz; (b) means for +measuring resultant ER phases and peaks of a responsive signal emitted by said tissue, in which each peak represents a potential in-phase or out-of-phase ER relative to an established normal phase of such tissue, each ER defining a waveform in which out-of-phase ER is associated with a tissue abnormality; and (c) means for delivery and applying to said tissue a waveform substantially inverse to that of out-of-phase waveforms to thereby decrease or nullify ER peaks associated with said abnormality of tissue.

It is an object of the present invention to employ principles of ER for the analysis and relief of pain and correction of abnormalities of human tissue.

It is another object to provide a system to analyze and digitize normal ER patterns of specific tissues.

It is further object of the invention to correct abnormal ER patterns by applying a countervailing or neutralizing ER field spectra utilizing inductive sensors and means to apply ER patterns of an intensity from about 0.05 to about 50 watts.

It is further object to measure and analyze ER out-of-phase peak resonances associated with abnormal tissue function, chronic pain, and traumatic injuries of soft tissue.

It is further object to provide a system of the above type in which useful ER pattern information is measured at a trigger point, at or near a tissue dysfunction or pain site, and a counter-resonance pattern is applied to said site to realign phase shifted resonance patterns associated with the electrons of cells affected by an abnormal or pain condition.

It is a yet further object to provide a system of the above type which can be readily interfaced with existing electromedical technologies including, without limitation, CT Scan, MRI, stereotactic imaging, and PET scanning. The advantage of this interface is the ability to visually illuminate an anatomical area with phase shift activity. By using an algorithm to monitor the amplitude of the signal and the degree of electromagnetic field or wave variation, various colors can be assigned to this phenomenon indicating antiphase or phase shifted areas. i.e., red being a reactive area and blue being a normal area. This information can then overlay a two or three dimensional diagnostic image, visually pinpointing an antiphase or phase shifted anatomical area. The advantage of this is to visualize functional variations and abnormalities as well as gross anatomical abnormalities.

Another iteration of the technology is the attachment of an LCD screen to the back of a hand held wireless induction coil. Anatomical areas of the body can be scanned while watching for color changes on the attached LCD when a phase shift area is detected.

The above and other objects in advantage of the present invention will become apparent from the hereinafter set forth. Brief Description of the Drawings and Detailed Description of the Invention and Claims appended herewith.

### BRIEF DESCRIPTION OF THE DRAWINGS

FIG. 1 is a block diagram showing the basic hardware and software functions of the inventive system.

FIG. 1A is a schematic view of a waveguide and control circuit used in the patient stimulation module.

FIG. 2 is a schematic view of a patient treatment unit (“PTU”) and associated diagnostic unit.

FIG. 3 is a block diagram view of a functional management unit (“FMU”).

FIG. 4 is a block diagram view of the local controller and system custom software operating upon a PC platform to control the PTU and manage a patient list. Also shown is a radio interface unit between the PC and the PTU.

FIG. 5 is a block diagram view of a tissue measurement module.

FIGS. 5A-5D show impedance, power and frequency relationships for the PTU.

FIG. 6 is a block diagram view of a communication module.

FIG. 7 is a block diagram view of the PTU inclusive of the PC radio interface and local controls of the PTU.

FIG. 8 is a block diagram view of the stimuli module.

FIGS. 9 and 10 are a respective signal and resonance peak waveforms of a healthy tissue.

FIGS. 11 and 12 are respective signal and spectrum waveforms of an abnormal tissue.

FIGS. 13 and 14 are respective signal and ER peak for spectra diagrams showing the treatment wave superimposed upon the waveform to be treated.

### DETAILED DESCRIPTION OF THE INVENTION

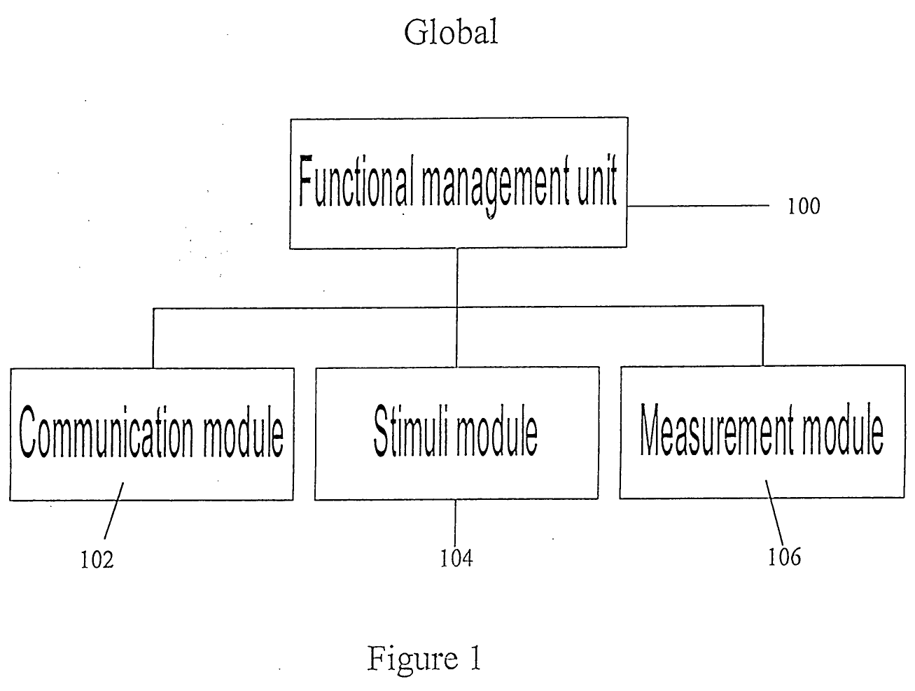

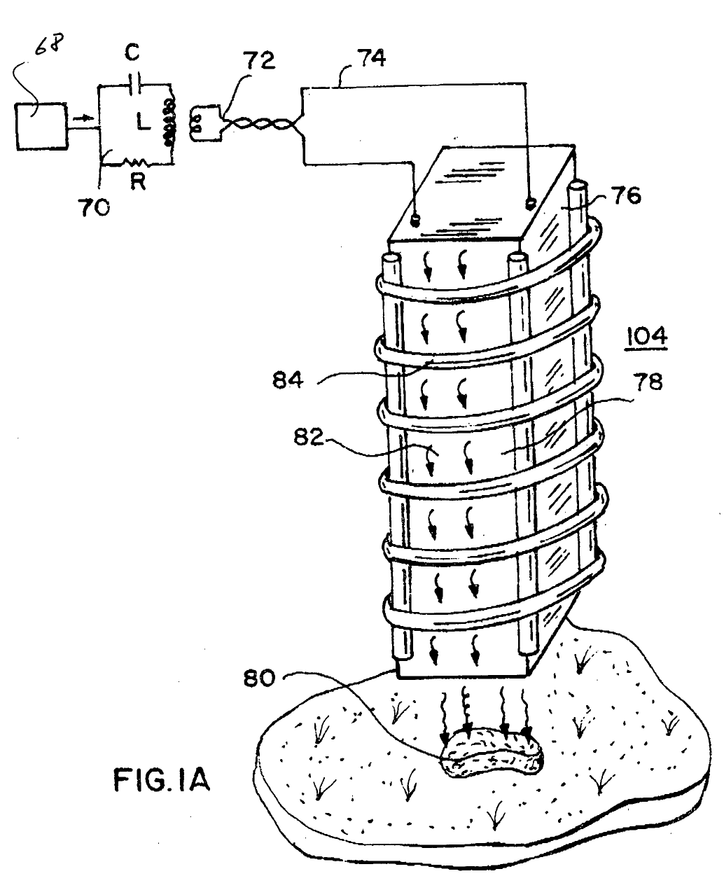

With reference to the general block diagram view of FIG. 1, my inventive system for analyzing and treating abnormality of human and animal tissues may be seen to include a functional management unit (“FMU”) 100 which supervises functions of a communication module 102, a stimuli module 104, and a measurement module 106.

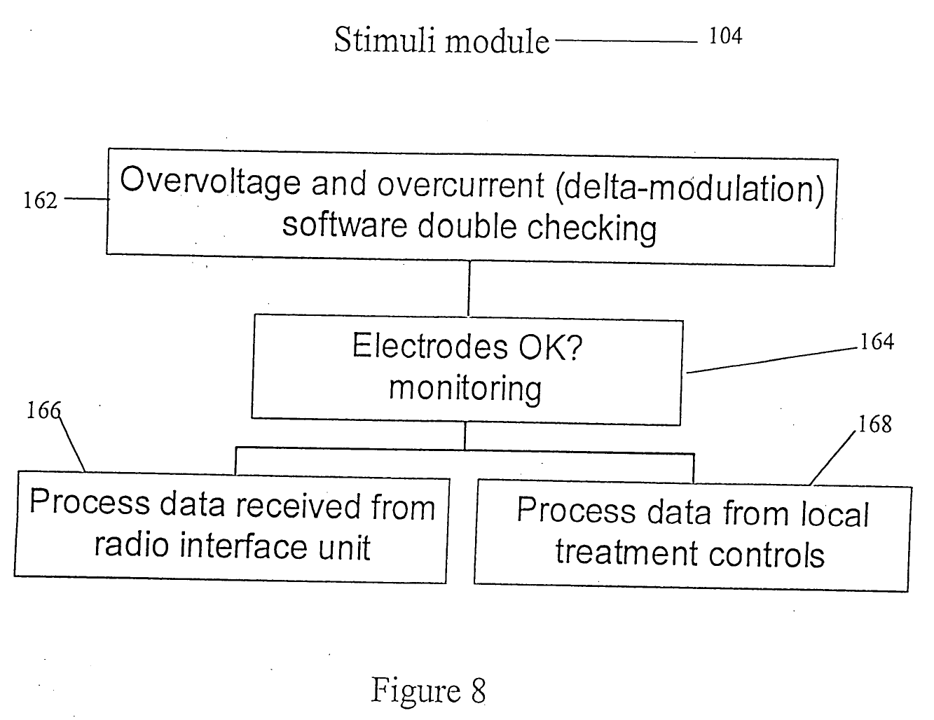

In FIG. 1A, the stimuli module 104 is shown in generic form. It may therein be seen to include a power supply 68 that supports an LC oscillator 70 which is coupled to a transmission line 72 which carries an oscillating, typically sinusoidal, current to an antenna 74. The EM emission thereof may then be transmitted axially into a waveguide 76 to produce traveling EM waves therein which comprise a form of energy transport that, including the so-called Poynting vector. An EM wave incident on an object in waveguide 76 will produce a radiation pressure on tissue 80. More importantly, through adjustments of oscillator 70, the LC resonant frequency and reflected waveform of tissue 80 can be determined, as well as the phase between resonant waveforms, as they exist in a healthy tissue of a category of interest. See FIGS. 9-10.

Shown externally upon waveguide 76 are coils 82 and capacitance plates 84 that may be used, with oscillator 70, to generate a “correction signal,” more fully described below.

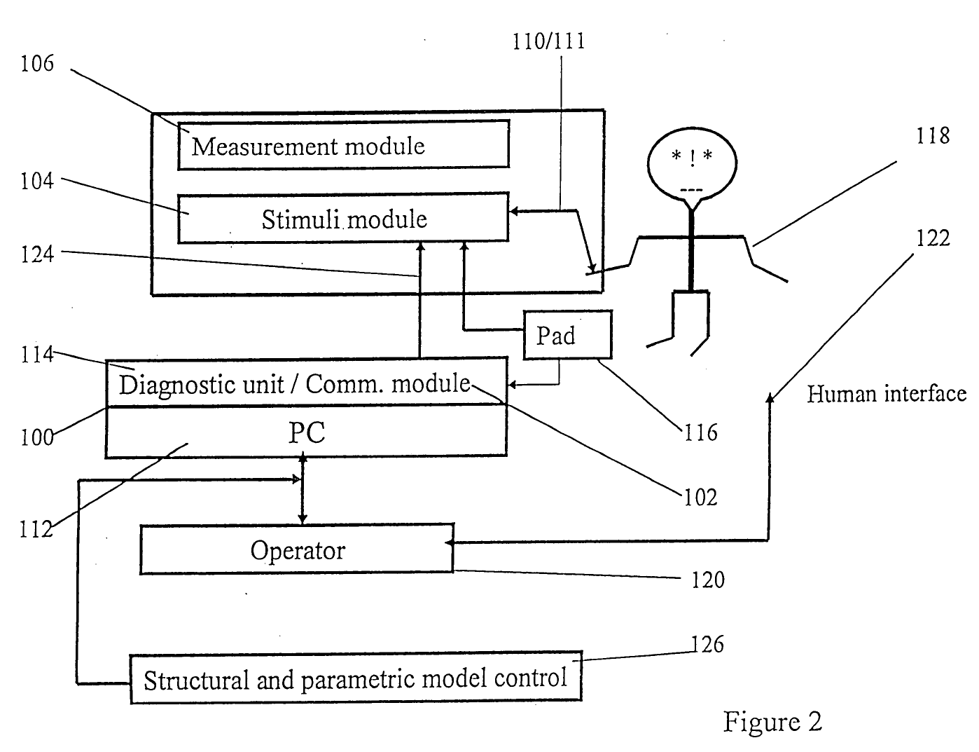

As may be noted in FIG. 2, the primary hardware of the inventive system is associated with a patient treatment unit (“PTU”) 108 which includes said measurement module 106 and stimuli module 104. The stimuli module functions through probes or induction coils 110/111 by thru which the initial data required by measurement module 106 is captured. In FIG. 2 may also been seen the physical relationship between a PC 112 and a diagnostic unit 114 which includes said communication module 102. Diagnostic unit 114 and PC 112 comprise integral components of said FMU 100. Further shown in FIG. 2 are pads 116 which facilitate treatment of patient 118 by an operator 120. Line 122 represents a human and animal interface between patient 118 and operator 120 while line 124 represents a radio interface means between the PC and diagnostic unit 114 on the one hand, and the PTU 108 on the other. Structural and parametric heuristic control of diagnostic unit 114 and PTU 108 are indicated by line 126 of FIG. 2.

The electrical output specifications of PTU 108 are as follows:

| | |
|---|---|
| Power Supply | 115 VAC, 60 Hz |
| Maximum Power Consumption | 21 W |
| Output voltage | Range of normal use: 50-60 V |
| | Peak pulse amplitude: 120 V |
| Pulse Rate | 1-490 Pulses/second, ±6% |
| Pulse Duration | 0.34-0.74 millisecond |
| Output Current (maximum) | 8.9 milliamps |
| Maximum charge per pulse | 7 micro coulombs |
| Wave Form | Complex pulse trains: variable |
| | frequency, variable pulse width, |
| | AC-coupled rectangular pulse |

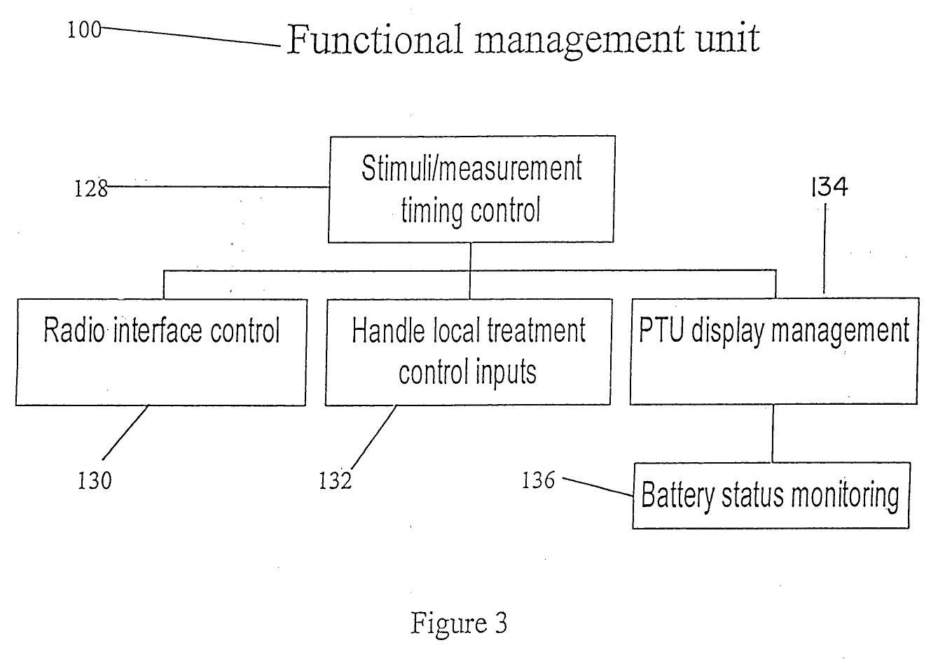

In FIG. 3 is shown FMU 100, inclusive of stimuli-measurement timing control means 128, radio interface control 130, local handle treatment control inputs 132 which are associated with said pads 116, a PTU display management facility 134 associated with said diagnostic unit 114, and a battery status monitor 136.

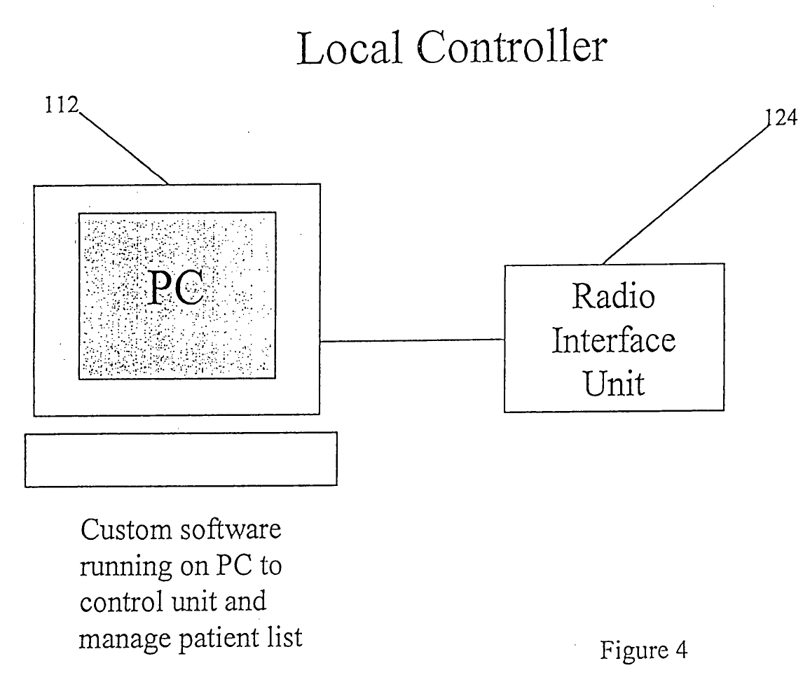

In FIG. 4 is schematically shown the use of custom software running upon said PC 112 to control the PTU 108 and manage a patient list. Said PC is connected to a PTU 108 through said radio interface 124.

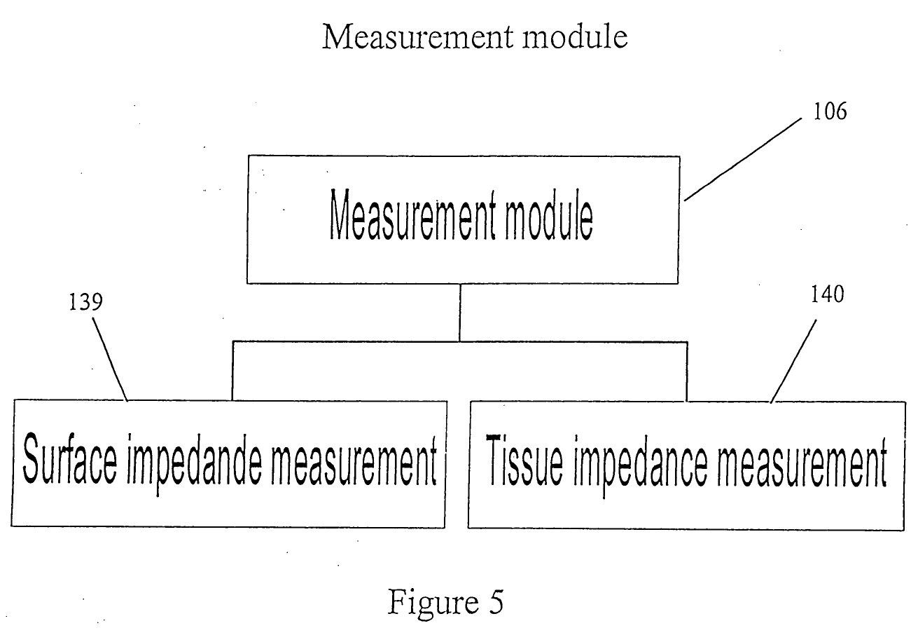

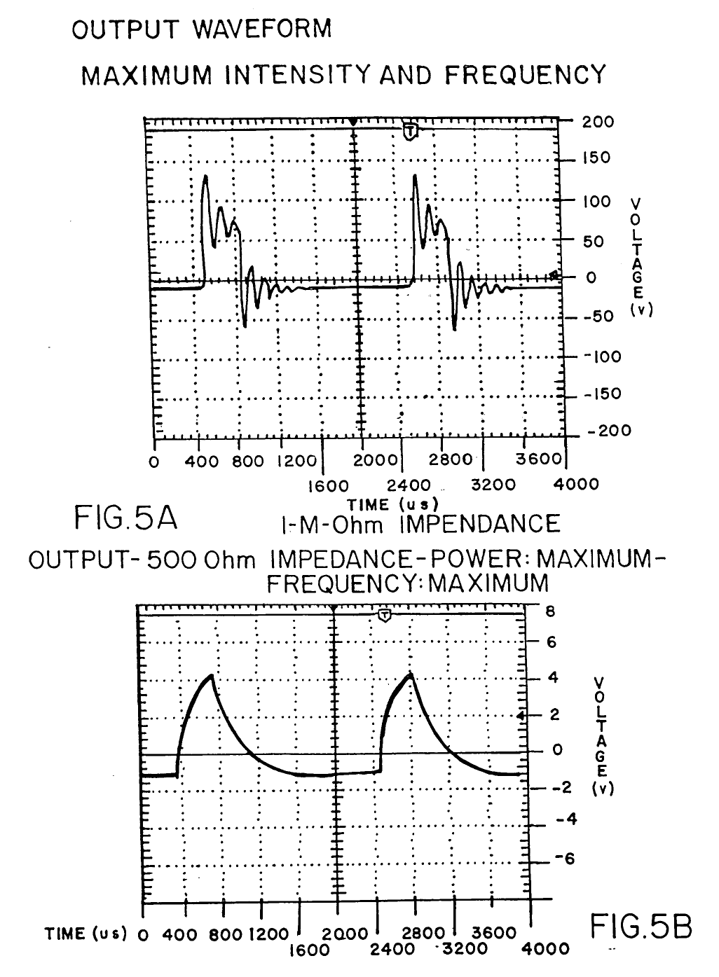

In FIG. 5 is shown measurement module 106 which includes means 138 for measurements of the surface impedance of a treated tissue and means 140 for measurement of the impedance of the tissue to be treated.

Output waveforms of PTU 108, showing various impedance, power, and frequency relationships are shown in FIGS. 5A-5D. More particularly, FIG. 5A indicates a 1 M-Ohm maximum impedance, in which the output waveform varies depending on the load as shown in FIGS. 5B-5D. That is, FIG. 5B shows voltage versus time at 500 ohms. FIG. 5C shows voltage versus time at 5000 ohms, and FIG. 5D shows voltage versus time at 10,000 ohms. Therein, changes in load affect both pulse duration and maximum pulse frequency. Maximum pulse frequency lies in a range of 490±6% from 500 ohms to 1,000,000 ohms. Lower impedances have lower maximum pulse rates. Pulse width is fixed at a given impedance, and declines from 0.74 milliseconds at 500 ohms to 0.34 milliseconds at 1,000,000 ohms.

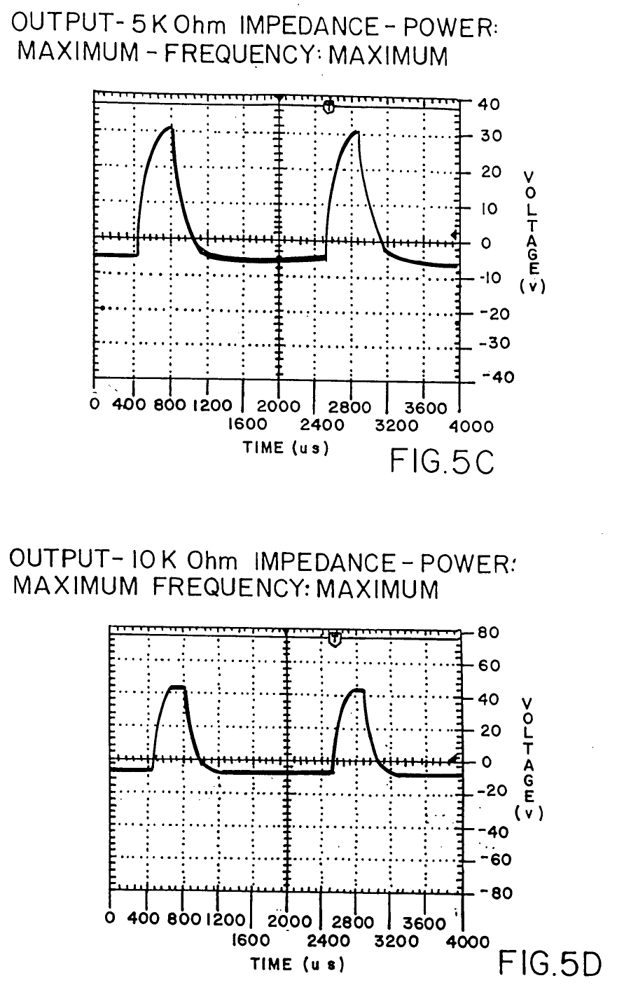

In FIG. 6 is shown communication module 102 and its important internal functions which include subsystem 142 which indicates the receipt and resending of error check stimuli information from a local controller (LC) which includes battery status information 144 and electrode or induction coil status information 146. Communication module 102 also includes subsystem 148 which sends, receives and error checks measurements of both surface and tissue as above described with reference to FIG. 5. Therein the skin-electrode or induction coil and tissue electrode or induction coil impedance is continually monitored at the LC in visual and/or audio terms to thereby enable the medical technician to adjust the pressure of the electrodes or the medium (such as electro-jelly) used between the electrode and the treated tissue.

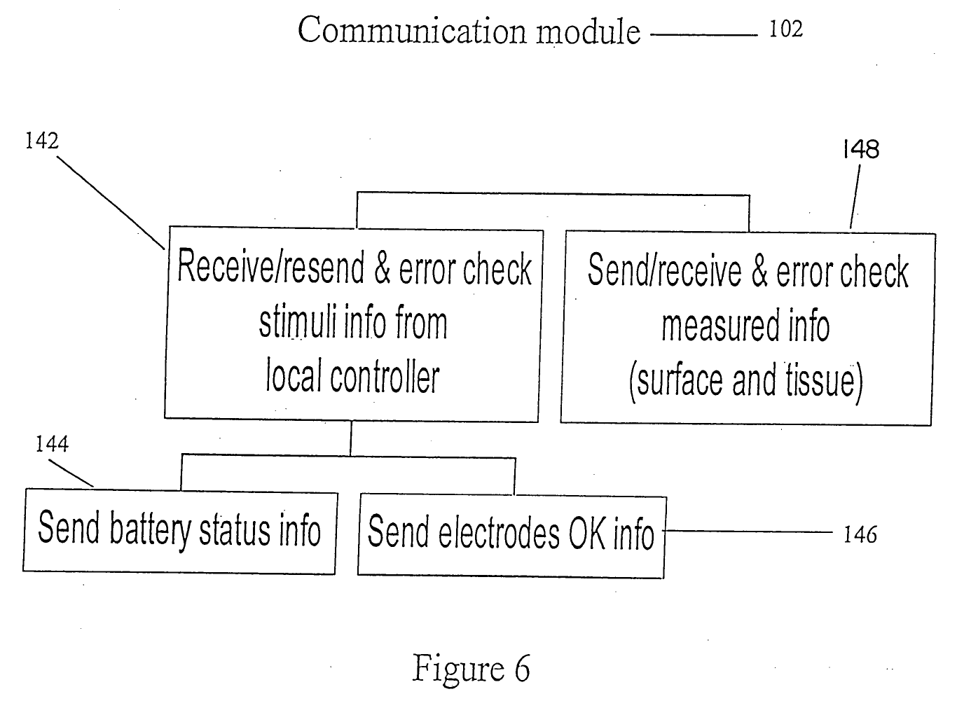

In FIG. 7 are shown the primary constituent subsystems of the PTU 108, these including a microcontroller 149 having a said local treatment controls 132, said display 134, status LEDs 135, a memory 150 used for purposes of recording data, and a DC to DC converter 152. As may be noted, the output of converter 152 feeds into pulse generator and level shifting means 154 which include current and voltage limiting means. The output of said means 154 is provided to means 156 for the simultaneous sensing of voltage and current associated with skin and tissue measurements. The output thereof is provided to said microcontroller 149 which operates with PC 112 through radio interface unit 124. The PTU 108 also includes a battery pack 158 and its charger 160.

Inputs to probes or induction coils 110 and 111 are provided through said dual voltage and current sensing means 156. It is noted that there are two areas in which magnetic resonance fluxuation is measured. The first is through an induction coil and the second is through the treatment measurement probes. The more phase shift (disorder or electrons loss of energy etc) the lower the measured amplitude and the greater the electromagnetic fluxuation.

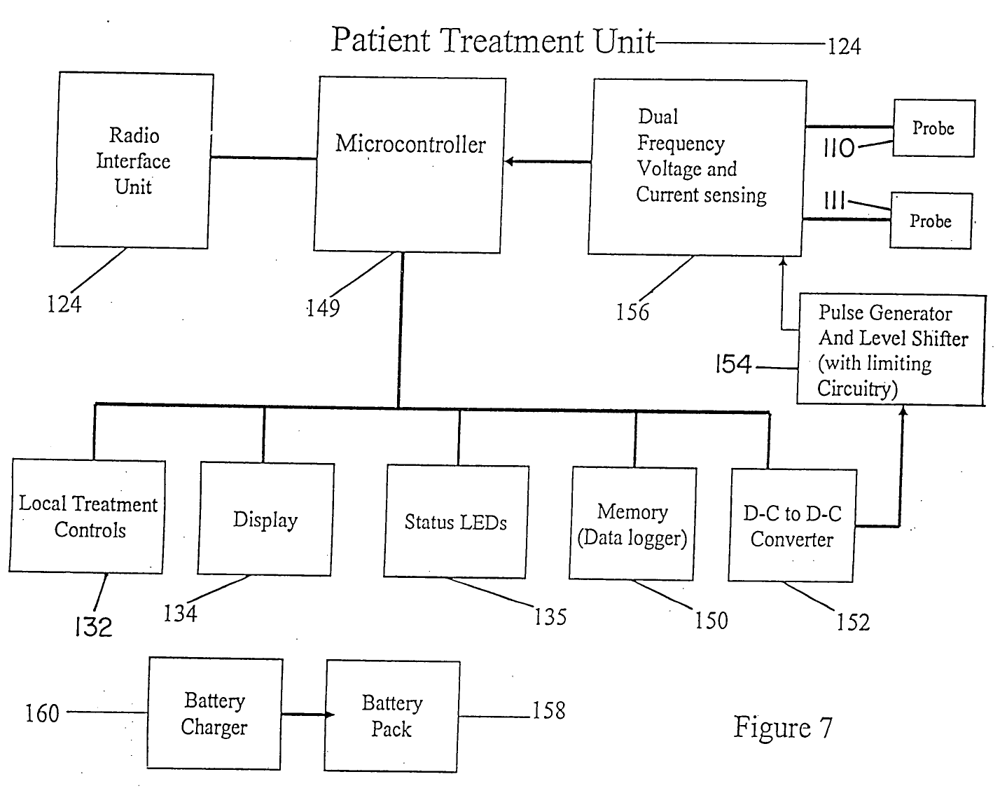

In FIG. 8 is shown stimuli module 104 and, more particularly, over voltage and over-current software monitoring means 162, associated electrode or induction coil monitoring means 164, and associated RI means 166 for processing data received from radio interface unit 124, and means 168 for processing data from local treatment controls 132.

It is to be appreciated that electrodes associated with probes 110/111 and pad 116, that is, two electrodes connected via wire, one of which electrodes is provide with a linear potentiometer are used to adjust or select the intensity of the energy provided to the treated tissue. A number of safety features are incorporated into the instant system including visual and/or audio warning means, amplitude limit means (per block 156), amplitude override means, amplitude ramp back means, and patient control means. Therein data transmitted from functional management unit 100 to the PTU 108 includes stimuli frequency, stimuli duty cycle, and patient pain threshold information (based upon patient history) to thereby optimize PTU-side intensity settings. Data transmitted between the PTU and FMU include skin voltage, electromagnetic fluxuation and current (see FIG. 5), phase between skin and voltage current, tissue voltage and current, phase between tissue voltage, electromagnetic fluxuation and current, and stimulus on/off status (see FIG. 3).

Importantly, the local controller (see FIG. 4) of the present ER system employs various algorithms.

Perhaps most importantly, the LC of the ER system employs various algorithms, starting with a so called inverse wave form of the injury tissue as a first order basis of treatment, this to be followed by robust stochastic models to generate appropriate stimuli profiles to enable the FMU 100 to provide a sophisticated treatment or correction signal. Therein at least three models or algorithms are contemplated, these including the following:
sequential, adaptive self-learning method and implementation (for a single electrode pair);
block adaptive self-learning method and implementation (for an electrode array);
one and multi dimensional neural network-based controller algorithms;
sequential data autoregressive method and implementation (for a single electrode pair); and
block data autoregressive method and implementation (for an electrode array)

In addition, the filtering of the measurement module of the FMU eliminates error signals which typically appear as waveform ripples, to thereby enable generation of a correction or treatment signal from a self-learning multi-electrode PTU, thereby having enhanced efficacy in the cancellation of pain and, ultimately, long term treatment of the condition of interest.

Combinations of algorithms may be employed to generate interchannel waveform correlations to ensure convergence of the model analysis and promotion of its learning curve for the modeling of the tissue injury, treatment profiles and peak resonances associated therewith.

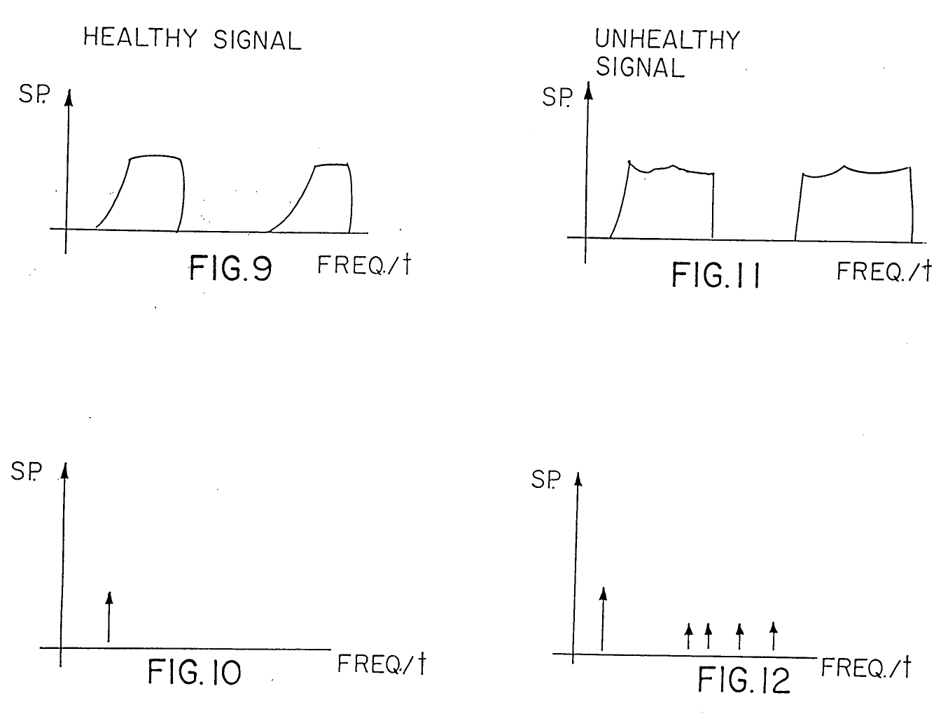

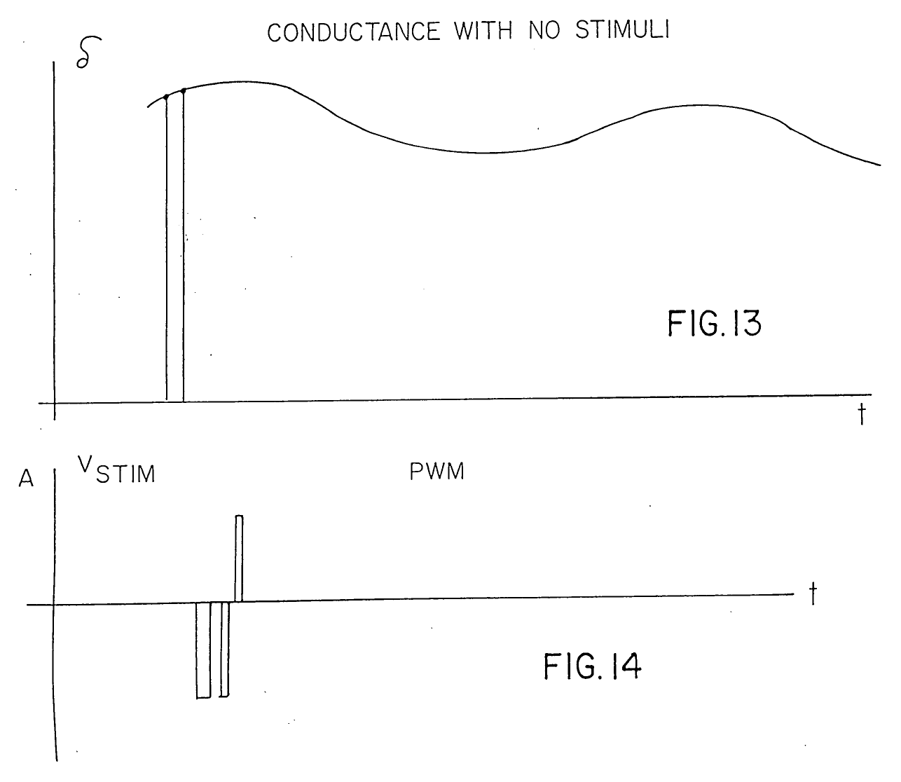

The next step is typically the generation of the inverse waveform or inverse ER spectra which is a generation of an opposite electrical resonance (ER) pattern from that shown in FIGS. 11 and 12. The application of this inverse pattern, has a pulse width modulation (PWM) process that is imposed upon a “sick” signal of the abnormal tissue is shown in FIG. 13. Thereby the system generates and applies to such tissue, a waveform of ER peak spectra substantially inverse to that of out-of-phase resonances of said tissue signal, to thereby increase or nullify ER peaks of the signal associated with abnormalities. See FIG. 14.

In summary, the technology employs a power of between about 0.5 to about 50 watts in treatment signals to increase, decrease, flatten or nullify out of phase resonance peaks of a measured waveform of the tissue to be treated. Similarly, the correction or treatment signal which is applied to treat the abnormal tissue signal obtained by the measurement module is intelligently developed by a self-learning multi-electrode PTU in which various heuristic algorithms, such as the above, are used to ensure convergence and efficient development of models necessary to optimize tissue profile, peak resonance codes, and the use of this information for effective therapy in an array of medical conditions.

This technology also enables treatment of conditions such as arthritis, post surgical pain, post surgical reduction of swelling inflammation and bruising, Osgood Schlater Disease, treatment of organ transplant patients for the purpose of reducing organ rejection, adhesive capsilitus, MS, ALS, motor neuron disease, reduction of keloid scaring treatment of skin graft sites for better vasculasation and better chance of successful graft improvement of circulation and oxygen saturation in compromised tissue and limbs, limb and digit reattachment for better chance of successful graft, improvement and normalization of conductivity in infarcted cardiac tissue, joint inflammation and injuries, fibromyglia, reflex sympathetic dystrophy, neuralgia, peripheral neuropathy, macular degeneration, wounds and sclerderma. However, a library of tissue profiles and peak resonance codes may be employed in the system in the development of a separate library of profiles and ER resonance codes for each patient and, also, as a baseline/or electromagnetic structures, of healthy tissue of many types, which might be employed in the generation of an inverse waveform (see discussion in FIGS. 13-14 below) or treatment purposes. Accordingly, my historic library of tissue profiles and peak resonance codes may be intergrated into the stochastic models, as set forth above, to generate appropriate stimuli profiles to enable a sophisticated treatment or correction signal. Therein a simple low-order low pass filtering process, to eliminate signal ripples, constitutes a starting point.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention.

1. A system for use of electrical resonant frequencies in analyzing and treating abnormality of human and animal tissue, the system comprising:
(a) means for generating an EM wave having a selectable ratio of capacitance to inductance, modulated in a selectable electrical resonance (ER) frequency relative to said tissue, the tissue to be treated across an EM spectrum of between about 10 Hz and about 500 MHz;
(b) means for measuring resultant ER phases and peaks of a responsive signal emitted by said tissue, in which each peak represents a potential in-phase or out-of-phase ER relative to an established normal phase of such tissue, each ER defining a waveform in which out-of-phase ER is associated with a tissue abnormality; and
(c) means for delivery and applying to said tissue a waveform substantially inverse to that of out-of-phase waveforms to thereby decrease or nullify ER peaks associated with said abnormality of tissue.

2. The system as recited in claim 1, in which said means of delivery comprises:
a waveguide by which said EM wave is delivered to the tissue as a traveling wave.

3. The system as recited in claim 1, in which said means of delivery comprises:
a solenoid which carries and controls inductance values of said delivered EM wave.

4. The system as recited in claim 1, in which said means for generating said inverse waveforms comprises:
an hollow elongate capacitor surrounded by a solenoid through which said inverse waveforms may be delivered

5. The system as recited in claim 1, in which said means of generating an EM wave comprises an LC oscillator.

6. The system as recited in claim 2, in which said means of generating an EM wave further comprises an LC oscillator.

7. The system as recited in claim 3, in which said means of generating an EM wave comprises an LC oscillator.

8. The system as recited in claim 4, in which said means of generating an EM wave comprises an LC oscillator.
